(19) United States
(12) Patent Application Publication
(10) Pub. No.: US 2017/0333593 A1
(43) Pub. Date: Nov. 23, 2017

(54) PRODUCTS AND DEVICES FOR CONTROLLING AND STOPPING BLEEDING AND METHODS OF USING

(71) Applicant: Cellphire, Inc., Rockville, MD (US)

(72) Inventors: Stephen H WILLARD, Chevy Chase, MD (US); Walt PEBLEY, Corvallis, OR (US)

(73) Assignee: Cellphire, Inc., Rockville, MD (US)

(21) Appl. No.: 15/525,689

(22) PCT Filed: Nov. 13, 2015
(86) PCT No.: PCT/US2015/060553

§ 371 Date: May 10, 2017

Related U.S. Application Data
(60) Provisional application No. 62/080,222, filed on Nov. 14, 2014.

(51) Int. Cl. A61L 24/00; A61M 11/00; A61K 35/19; A61K 9/19

(52) U.S. Cl. CPC A61L 24/0005; A61L 24/0015; A61K 9/19; A61M 11/006; A61K 35/19; A61L 2300/64; A61L 2400/04

(57) ABSTRACT

Systems, devices, compositions, and methods for treating bleeding, for containing blood that has been lost by a subject due to bleeding, and for treating surfaces contaminated with blood or expected to be contaminated with blood are disclosed. The systems, devices, compositions, and methods utilize platelets, platelet-derived materials, or both. The invention includes sprayers, nebulizers, and equivalents, to deliver platelets and/or platelet-derived materials to desired surfaces. Exemplary embodiments include the use of a pressurized sprayer configured to deliver a topical administration of a lyophilized platelet and/or platelet-derived material to a surface.

### CROSS-REFERENCE TO RELATED APPLICATIONS

This application relies on the disclosure of, and claims the benefit of the filing date of U.S. provisional patent application No. 62/080,222, filed 14 Nov. 2014, the entire disclosure of which is incorporated herein by reference.

### BACKGROUND OF THE INVENTION

### Field of the Invention

The present invention relates to the field of medicine. More specifically, the present invention relates to compositions, devices, and methods for administering a composition of matter that improves healing and aids in protection from blood, blood products, and blood-borne diseases.

### Discussion of Related Art

Platelets are formed in the bone marrow as fragments of megakaryocytes. In the general blood circulation, they are biconvex discoid-shaped non-nucleated bodies that are present at a concentration of 150,000-450,000 per microliter (μl). Platelets play a crucial role in hemostasis, and they are the first line of defense against blood escaping from injured blood vessels. When bleeding from a wound suddenly occurs, the platelets gather at the wound and attempt to block the blood flow by initiating formation of a clot. There are two general mechanisms to clot formation. In one mechanism, a clot begins to form when the blood is exposed to air. The platelets react to the presence of air to initiate formation of fibrin. The resulting fibrin forms a web-like mesh that traps blood components within it. In the other general mechanism, damaged blood vessels release signals that increase the stickiness of platelets in the area of the injury. The sticky platelets adhere to the damaged area and quickly form a hemostatic plug. At the same time, the platelets release a series of signals that prompt clotting factors in the blood to form fibrin to reinforce the platelet plug. Between the platelet and its reinforcements, a sturdy clot is created that acts as a patch while the damaged area heals.

Platelets contain a number of important growth factors within their alpha granules that contribute to the process of hemostasis and wound healing. Studies have found that growth factors, such as platelet derived wound healing factors (PDWHF), platelet-derived growth factor (PDGF), transforming growth factor (TGF), and insulin growth factors (IGF), among others, are important in different stages of the wound healing cascade and greatly influence mitogenic and cellular differentiation activities. It has also been shown that growth factors help cells to localize to the area of a wound.

Bleeding, especially uncontrolled bleeding, can produce many undesirable outcomes. Indeed, even spilled blood or its residue can present medical concerns. Bleeding can increase the transmission of communicable diseases, increase an injured person's likelihood of infection, weaken a person, and ultimately cause fatal bleed out of a human or animal.

Compressible bleeding has traditionally been treated with topical solutions, such as bandages, antibiotics, and hydrogels. There are disadvantages to these materials in that they do not augment the body's natural responses, such as hemostasis, to stop internal bleeding. Patients with clinically significant hemostasis defects often continue to bleed in spite of compression of the wound site with these materials. These conventional treatments do not stop bleeding fast enough, nor do they start healing quickly enough, and their removal may re-open the wound surface. Conventional methods also often require repeated reapplication and other forms of direct intervention. These conventional methods additionally require direct contact between an interventionist, such as a nurse or doctor, and blood.

In many situations, contact with blood is highly undesirable. This concern is often true even when wearing protective clothing. For example, patients infected with a hemorrhagic fever virus, such as the Ebola virus, may bleed from their eyes, nose, and extremities. The virus can also be shed into the alimentary canal, urine, sweat, mucus, and other bodily fluids. Ebola patients, for example, present a particularly dangerous situation to caregivers because the virus is transmitted by blood or other bodily fluids. In particular, caregivers are at a very high risk when removing their protective clothing. By avoiding actual contact with an Ebola victim, a caregiver can avoid being exposed to any bodily fluids, including blood, while still treating the patient. Conventional treatments do not allow a caregiver to maintain a safe distance while containing blood from a patient (e.g., applying compressive force). Similar issues arise for other fevers from different RNA virus families, such as the Arenaviridae, Filoviridae, Bunyaviridae, Flaviviridae, and Rhabdoviridae families.

Patients that have suffered severe burns are also in need of wound treatment. Conventional treatments, such as bandaging, have notable drawbacks, including being extremely painful due to re-injury or tissue damage during changing of dressings, the need for repeated reapplication, and they often do not allow for adequate isolation of the wound from the air.

Slaughterhouse blood also presents a situation in which blood should be carefully contained. For example, bovine spongiform encephalopathy, or mad cow disease, is transmitted through misfolded proteins that might be present in contaminated blood. Conventional methods for containing slaughterhouse blood include draining the blood into large pits. However, there is still a need for safe disposal of the collected blood.

These problems have highlighted the need of improved systems for collecting blood along with aiding in wound treatment and healing. Likewise, the problems indicate that there is a need for methods of treating wounds and containing infectious diseases using systems and methods that promote caregiver safety while improving the treatments of wounds. Finally, there continues to exist a need to form a barrier between healthy people, such as health care workers, and blood or bodily fluids from an injured or sick person. This barrier can be on the patient, the caregiver, or the bodily fluids when a single physical treatment with a chemical, such as chlorine bleach, is not possible or advisable.

### SUMMARY OF THE INVENTION

One aspect of the present invention provides a system for delivering a protective composition to a person in need, or suspecting of being in need, of protection from a biological material. The system generally comprises a composition that provides protection from the biological material, and a device for delivering the composition. The composition comprises platelets or platelet-derived material. The composition can also comprise an application material to assist in delivering and/or maintaining the platelets or platelet-derived material at the desired site on the body of the subject. Such a combination is referred to herein as an application mixture. While not so limited, in exemplary embodiments, the device is one that is capable of spraying, aerosolizing, nebulizing, etc. the composition or application mixture of the invention. Typically, the biological material from which protection is needed is one that contains blood, a blood product, a component of blood, or an infectious agent present in these.

Another aspect of the invention is a method for delivering a protective composition to a person in need, or suspecting of being in need, of protection from a biological material. In general, the act of delivering can be any suitable action that results in the protective composition of the invention coming into contact with the person as a result of use of the device of the invention. Where the person is actively shedding a bodily fluid that contains blood, a blood product, or a component of blood, preferably the method includes delivering the protective composition to the site of shedding. The method can be a therapeutic method, but is preferably practiced as a prophylactic method of protecting a person prior to coming in contact with the biological material. However, the method of prophylaxis is not limited to treating the person believed to be coming in contact with the biological material. Rather, it can be a method practiced on the person shedding the biological material in order to protect the person coming in contact with that person. The method can also be practiced to stop the spread of a disease by a subject by stopping shedding of the infectious agent by the subject.

### BRIEF DESCRIPTION OF THE DRAWINGS

The accompanying drawings, which are incorporated in and constitute a part of this specification, illustrate several embodiments of the invention, and together with the written description, serve to explain certain principles of the invention. The embodiments depicted in the drawings should not be interpreted in limiting the scope of the invention in any way.

FIG. 1 is a flow diagram of a configuration of an exemplary embodiment of a sprayer system according to the invention.

FIG. 2 is a flow diagram of an alternative configuration of an exemplary embodiment of a sprayer system according to the invention.

FIG. 3 is a flow diagram of an alternative configuration of an exemplary embodiment of a sprayer system according to the invention.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention, as broadly disclosed herein. Rather, the following discussion is provided to give the reader a more detailed understanding of certain aspects and features of the invention.

Before embodiments of the present invention are described in detail, it is to be understood that the terminology used herein is for the purpose of describing particular embodiments only, and is not intended to be limiting. Further, where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention.

Unless defined otherwise, all technical and scientific terms used herein have the same meaning as commonly understood by one of ordinary skill in the art to which the term belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a platelet” includes a plurality of such platelets and reference to “a saccharide” includes reference to one or more saccharides, and equivalents thereof known to those skilled in the art, and so forth. Furthermore, the use of terms that can be described using equivalent terms include the use of those equivalent terms. Thus, for example, the use of the term “subject” is to be understood to include the terms “patient”, “person”, “animal”, “human”, and other terms used in the art to indicate one who is subject to a medical treatment. As another example, the use of the term “blood” is to be understood to include the terms “plasma”, “blood product”, “platelet rich plasma”, and other terms used in the art to indicate whole blood or components of whole blood. The use of multiple terms to encompass a single concept is not to be construed as limiting the concept to only those terms used.

Systems and methods are described herein to effectively stop bleeding or contain released or discharged blood or bodily fluids that comprise blood. Generally speaking, when the term blood is used in this document, it is to be understood that other bodily fluids, such as saliva, tears, mucous, urine, etc. may also be present with the blood. In exemplary embodiments discussed in detail herein, the systems and methods are directed toward the closure and healing of wounds, or to otherwise stop the loss of bodily fluids that contain blood. In some embodiments, the systems and methods may be used in conjunction with other devices than those detailed herein. More specifically, the exemplary embodiments described herein relate to spray studies for illustrative purposes only. The systems and methods described herein may be used for many different industries and purposes, including not only human and animal therapeutic and prophylactic purposes but other industries completely. In particular, the systems and methods may be used for any industry or purpose for protection from blood or blood products, coagulation of blood, or control/cleanup of blood or other bodily fluids, without direct application of the compositions of the invention to a living subject. For multi-step processes or methods, steps may be performed by one or more different parties while still qualifying as practicing the present invention.

As mentioned above, an aspect of the invention is a system for delivery of a composition to a subject for the protection of that subject from blood or a blood-borne disease. In its basic form, the system comprises: i) a composition that comprises platelets or platelet-derived material; and ii) a device configured to deliver the composition to a site on the subject's body. As discussed below, the system can comprise additional elements. In an exemplary embodiment, the system is a system for applying platelets or platelet-derived material to a surface, where the system comprises a device comprising, in physical and fluid connection, a pressure delivery unit, a composition container, an application material container, and an application unit; wherein the composition container contains a composition comprising the platelets or platelet-derived material, and wherein the application material container contains application material that improves the delivery, maintenance, or both, of the platelets or platelet-derived material at the desired site on the surface.

The composition of the invention can include numerous substances. However, in its basic form, it includes platelets and/or platelet-derived material. Platelet-derived material includes, but is not limited to, lyophilized (freeze-dried) platelets, rehydrated lyophilized platelets, platelet microparticles, platelet membranes, platelet alpha granules, and platelet dense granules.

In some embodiments, the composition is in the form of a dry powder, which can be isolated from blood and preserved as described in U.S. Pat. No. 7,811,558, U.S. Pat. No. 8,097,403, or U.S. Pat. No. 4,994,367, which are incorporated herein by reference in their entireties. In embodiments relating to powdered compositions, the compositions can be prepared by lyophilization. In certain methods, components of the composition can be chemically fixed or cross-linked. In certain embodiments, the platelet material may be derived using recombinant methods.

One feature of certain embodiments of the composition of the invention is that platelet microparticles and other platelet-derived particles can accelerate clot formation, likely at least in part by way of their ability to promote tenase and prothrombinase clotting factor activities, thereby enhancing thrombin-generating capacity and promoting rapid clot development at the bleeding site. In addition, due to the fact that the compositions can comprise a platelet-derived material and can contain a number of important growth factors, they can also contribute to the process of wound healing and tissue regeneration. Mitogenic lipids and growth factors, such as platelet-derived wound healing factors (PDWHF), platelet-derived growth factor (PDGF), transforming growth factor (TGF), and insulin growth factors (IGF), among others, are important in different stages of the wound-healing cascade and greatly influence mitogenic and cellular differentiation activities. Thus, in some embodiments, one or more of these factors can be included in the composition.

The platelets that form the source of the platelets and platelet-derived material of the compositions may be from any source. Accordingly, they may be from an animal, such as an equine, a canine, an ovine, a porcine, a feline, a bovidae, a lagomorpha, a rodent, or a primate. In preferred embodiments, the source of the platelets is human. In certain cases, the platelets may be provided as a mixture from two or more sources, such as a mixture of two or more units of blood from the same species, preferably three to five, more preferably six to 10 or more units obtained from random blood donors to a public blood bank. The platelets may be provided from a fresh source (i.e., in-dated platelets from blood obtained from a donor less than six days prior to production or use of the composition), although out-dated platelets may be used in some situations, particularly for preparation of lyophilized platelets intended for use as a hemostat to aid in stopping bleeding at a particular site of injury.

As discussed above, in embodiments, the composition may be a powder. It is to be understood that the composition may also be a liquid, a paste, or a gel. In one embodiment, the composition can be a hydrogel mixture, which can, but is not necessarily, added to a wound dressing. In an embodiment, a wound dressing may take the form of a collagen dressing and may include layered hyrdogel composition. In certain other embodiments, the composition can be added to a hydrophilic mixture, such as petroleum jelly or mineral oil. It can be applied in any manner used to coat materials.

The particle size of the platelet material of the composition is not particularly limited and it may be in the range of 5-100 percent passing a 500, 400, 270, 100, 80, 50, or 30 mesh screen. In general embodiments, the average particle size of the platelet material of the composition is less than the average particle size of fresh platelets. For example, while the average size of fresh platelets (longest axis) might be two to three micrometers, the average size of the platelets of the composition (longest axis) can be below two micrometers.

The composition can comprise one or more salts, such as phosphate salts, sodium salts, potassium salts, calcium salts, magnesium salts, and the like. The composition can also include sugars, such as monosaccharides and disaccharides (e.g., maltose, dextrose, mannose, trehalose, sucrose, polymers of sucrose, glucose); polysugars, such as Ficoll-70 and Ficoll-400; glycerol; triglycerides; polysaccharides; lipids; dextran; polyvinyl pyrolidine (PVP); starch; hydroxyethyl starch (HES); and the like. Yet other exemplary substances include biological molecules derived from human or animal sources, such as polypeptides (e.g., albumins such as bovine serum albumin and human serum albumin), casein, laminin, fibrinogen, and the like.

One particular group of substances that may be present in a composition of the invention is chemical and biological compounds that function as active pharmaceutical ingredients (e.g., antibiotics, antivirals, antifungals, other disinfectants, and like therapeutics). Other substances may be compounds that function as markers or reporter molecules, including contrast agents for certain medical procedures.

Compositions included in embodiments may contain fibrin. Compositions according to embodiments that do not contain fibrin may provide an advantage over compositions known in the art, for example when the embodiments are used to treat non-compressible wounds.

In embodiments, compositions comprise platelets, platelet-derived substance, or both, but no other substance that is biologically active in forming a clot. In preferred embodiments, the platelets and platelet-derived substances provide some or all of the biological characteristics and biochemical activities as fresh, normal platelets. Preferably, compositions of the invention comprise platelets and/or platelet-derived substances that are sufficiently similar to fresh, normal platelets to provide biological and biochemical activities that are sufficient to initiate clot formation and/or reduction or cessation of unwanted bleeding.

Where the composition comprises substances other than platelets and/or platelet-derived material, preferably, these substances are compatible with the function of platelets or aid in retention of platelets at a site of injury.

One such substance is a buffer that maintains a liquid form of the composition at a stable pH (whether for a period of time prior to delivery or for a period of time after delivery). Where present, preferably the buffer, when in a liquid composition, maintains the composition at a pH of about six to about eight. The buffering component may be any buffer that is non-toxic to platelets at the concentration used and provides adequate buffering capacity to the composition at the temperatures and volumes at which the composition will be exposed. Thus, the buffer may comprise any of the known biologically compatible buffers available commercially, such as HEPES, phosphate-buffered saline (PBS), and Tris-based buffers, such as TBS. Those of skill in the art are well aware of the numerous additional buffers that satisfy the criteria set forth herein. As such, an exhaustive listing is not required.

The composition of the invention can be mixed with an application material to form an application mixture. The application material is a substance or mixture of substances that is combined with the composition to enhance or otherwise affect the characteristics of the composition. Non-limiting characteristics include viscosity, tolerance to rapid changes in pressure, heat and cold tolerance (e.g., resistance to platelet activation after temperature changes), adhesion properties with respect to binding to animal skin, mucous membranes, etc., adhesion properties with respect to binding to wound coverings, and the like. In an embodiment, the composition is combined with an application material to create an application mixture that has a viscosity of about 1 to 800 cp. In certain embodiments, the viscosity may be adjusted to increase or decrease the velocity of exit from the device of the invention, which may allow for a different application distance to blood-containing substances on a patient or blood-containing substances on other surfaces, such as medical equipment. The viscosity may be configured to adjust the shearing properties of the platelets and/or platelet-derived material during application. Optimization of these parameters is well within the skill of those of ordinary skill in the art without undue or excessive experimentation.

In non-limiting embodiments, the application material is sterile water, blood or blood components (e.g., plasma), glycerol, saline, buffered saline, petroleum jelly, hydrogel, cellulose, hydroxy ethyl cellulose, hydroxy methyl cellulose, mineral oil, amyl acetate, benzalkonium chloride, castor oil, clove bud oil, ethyl alcohol, isobutane-propane (propellant), n-Butyl acetate, nitrocellulose, and combinations thereof.

In an embodiment, the composition may be mixed with the application material to give a certain platelet or platelet-derived material concentration and a certain viscosity. In an embodiment, the platelet or platelet-derived material concentration is in the range of about 0.1 to 3.0×10⁹ per mL. In an embodiment where the composition is combined with the application material, the final platelet or platelet-derived material concentration in the application mixture is in the range of about 0.01 to 2.0×10⁹ per mL. In an embodiment, the platelet or platelet-derived material concentration is in the range of about 1.0 to 30×10¹⁰ platelets or platelet-derived materials per square inch (about 6.45 square cm) after being administered to a surface, such as a patient's skin. In certain embodiments, the application mixture is in the form of a powder and the platelet or platelet-derived material concentration is adjusted to achieve a concentration of about 0.1 to 1.0×10⁹ per mL when the platelets or platelet-derived material are reconstituted by shed blood or other exudate.

In an embodiment where the platelet material is added to mineral oil, petroleum jelly, or other relatively viscous substance, a homogenizer, mixer, high-shear fluid processor, or an emulsifier may be used to create an emulsion. A microfluidizer may also be used to physically shear the platelets. In some embodiments, the pressure and flow rate of a spray apparatus may be utilized to shear the platelets during operation of a deliver device. In other embodiments, intact platelets may be preferred and utilized.

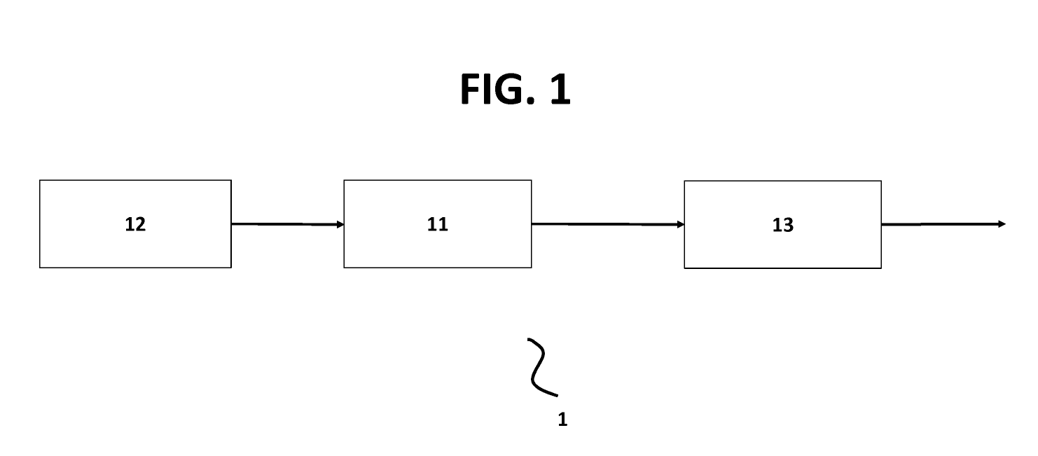

In addition to the composition and optional application mixture, the system of the invention includes a device for delivery of the composition and application mixture to a subject. The device will now be described in three exemplary configurations, although more will be immediately apparent to the ordinary artisan. In the first exemplary configuration, as depicted in FIG. 1, device 1 comprises container 11 to hold the composition prior to ejecting the composition from device 1. A pressure delivery unit 12 is physically connected to composition container 11 such that pressure developed by pressure delivery unit 12 is transferred to composition container 11. As such, it should be evident that the connection allows a fluid connection in which pneumatic/gas or liquid pressure is transferred from pressure delivery unit 12 to composition container 11. Composition container 11 is physically connected to application unit 13 such that pressure from composition container 11 forces the composition into application unit 13. As such, it should be evident that the connection allows a fluid connection in which pneumatic/gas or liquid pressure is transferred from composition container 11 to application unit 13. The force further causes the composition to exit application unit 13.

It is to be noted here that, in the following disclosure, to avoid repetition, the ordinary artisan is to understand that, where there is a disclosure of a physical connection between two units or elements of the device, there is also a pneumatic/gas or liquid connection by way of which pressure can be transmitted. In addition, it is to be understood that in the disclosure and claims of this document, unless otherwise noted, stated physical/fluid connection of certain elements/units with others does not imply connections with all other elements, but only those specifically referenced. For example, disclosure of a system for applying platelets or platelet-derived material to a surface in which the system comprises or includes a device comprising, in physical and fluid connection, a pressure delivery unit, a composition container, an application material container, and an application unit is not intended, and does not imply that all four elements are physically and directly connected to each other, but rather is intended to teach that there are embodiments in which the elements are connected in the order in which they are disclosed, or that variations in the order are encompassed.

Composition container 11, and all containers mentioned below, can be any structure that is suitable for containing the composition, application material, or any other substances. It thus can be a jar, vial, ampoule, etc. made of a relatively durable material, such as plastic, glass, or metal, which is configured to make an air-tight seal with other elements of the device (e.g., by a threaded end or by a suction seal via O-ring compression). Such configurations are well-known in the art and thus need not be detailed herein. Alternatively, it can be made of a relatively pliable, flexible material, such as a plastic bag, which includes a port or other access point for attaching the bag to other elements of the device. Again, such access points are well-known in the art and thus need not be detailed herein.

In preferred embodiments, a container is reversibly sealed to maintain the composition in a desired state (e.g., dry, sterile). Apart from the well-known serum vial design, which typically includes crimp-sealed stoppered caps that are easily punctured by sharp objects, such as needles, mention can be made of the following concepts. Sealed compositions can include a foil overwrap to protect from oxygen, moisture, or light, and which also might increase shelf stability. During preparation of a composition or application material, a vacuum chamber can be configured to include heating bars that can be positioned on the lower or upper portions of shelves or walls such that one or more edges of a bag (or other container) can be pushed together and sealed under vacuum. A bag or other container to be sealed can be comprised of foil, PVC, or other material capable of self-adhering when heat is applied. A bag or other sealed container can also be sealed utilizing an adhesive material. Embodiments of this aspect of the invention can include sterilizing the composition, for example by heat treatment or gamma irradiation treatment, before or after the composition is packaged in the container.

The composition and/or application material can be added to a container in any amount that is desirable. That is, the composition and/or application material can be present in a container in an amount for a single dose/application to a subject or in an amount for multiple applications (i.e., bulk packaging).

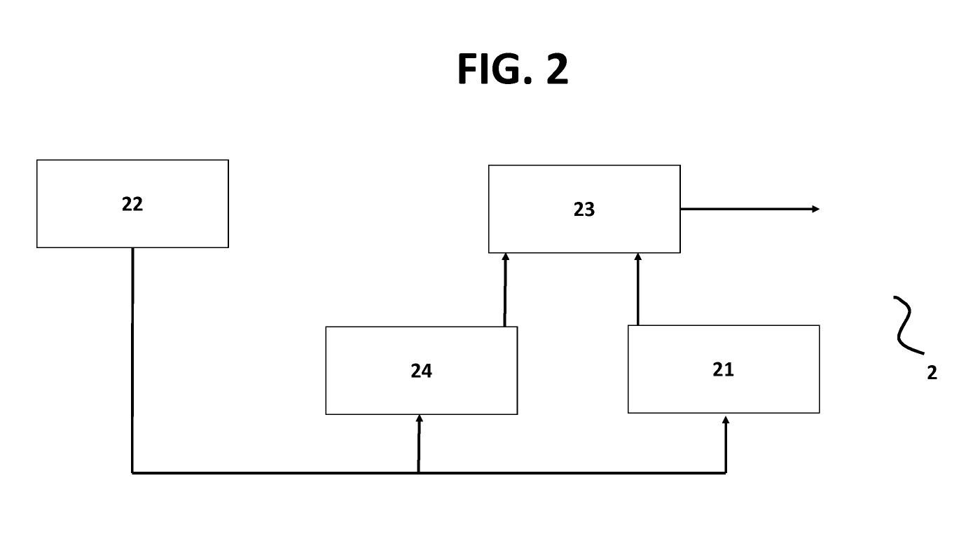

Another non-limiting exemplary configuration of a device according to the invention is depicted in FIG. 2. According to this configuration, device 2 comprises composition container 21 to hold the composition prior to ejecting the composition from device 2. A pressure delivery unit 22 is physically connected to composition container 21 and to an application material container 24, such that pressure developed by pressure delivery unit 22 is transferred to both composition container 21 and to application material container 24. Composition container 21 and application material container 24 are both physically connected to application unit 23 such that pressure from composition container 21 forces the composition into application unit 23 and pressure from application material container 24 forces the application material into application unit 23. In application unit 23, the composition and application material mix, and the mixture then exits application unit 23 as a result of the force imparted ultimately from pressure delivery unit 22.

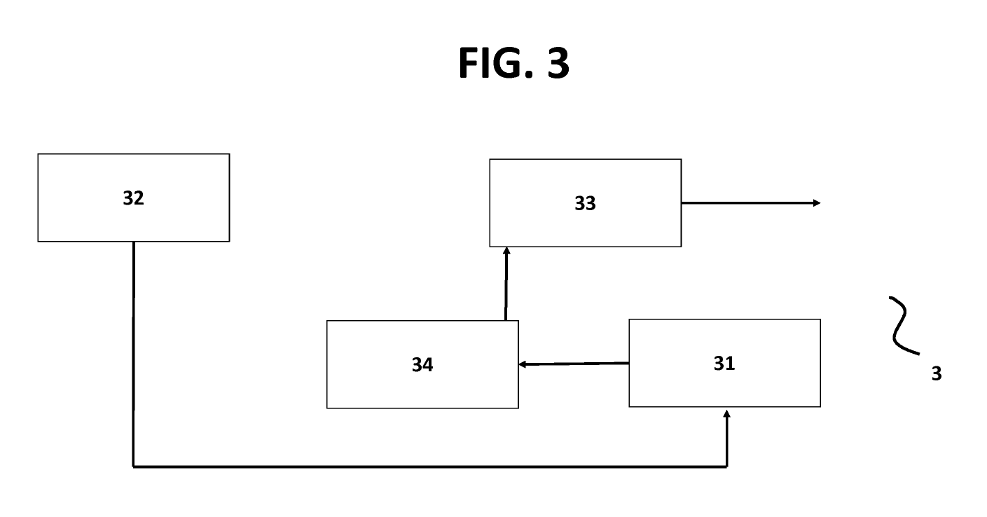

Yet another non-limiting exemplary configuration of a device according to the invention is depicted in FIG. 3. According to this configuration, device 3 comprises composition container 31 to hold the composition prior to ejecting the composition from device 3. A pressure delivery unit 32 is physically connected to composition container 31 such that pressure developed by pressure delivery unit 32 is transferred to composition container 31. Composition container 31 is physically connected to an application material container 34, such that pressure from composition container 31 forces the composition into application material container 34, where the composition mixes with application material, forming a mixture. Application material container 34 is physically connected to an application unit 33 such that pressure from application material container 34 forces the mixture into application unit 33. In application unit 33, the composition and application material may further mix. The mixture exits application unit 33 as a result of the force imparted ultimately from pressure delivery unit 32.

Those of ordinary skill in the spray gun art will immediately recognize other configurations for the device of the invention, and all such configurations are contemplated as part of this invention. Further, other configurations will be immediately apparent to those of ordinary skill in any art pertaining to spraying of materials. For example, other non-limiting configurations of the device include mechanical pump sprayers (e.g., for delivery of perfume by hand, for spraying lawn care products, for aerosolizing or nebulizing medical products, such as nose sprays) and pressurized containers (e.g., for delivery of spray paint).

In view of the discussion above, it will be apparent that in certain embodiments, the composition and/or application material may be housed in a container that is pressurized by a pump or compressor, where the type of pump or compressor is not particularly limited. In exemplary embodiments, a pressure delivery unit delivers pressure by way of a pneumatic pump, by way of a gas or liquid propellant, or by way of compressive force supplied by a human. In many embodiments, the pressure is adjusted to change the velocity of the spray along with adjusting the lysing characteristics of the platelets or platelet material. The operating pressure is not particularly limited and may be in the range of 0.1 to 50 bar, and more particularly in the range of about 0.5 to 10 bar. In certain embodiments, the pressure range can be adjusted to enhance the shear forces on platelet powder particles to enhance their break-up when rapid action is preferred.

Likewise, in view of the discussion above, it will be apparent that in some embodiments, the composition and/or application material may be housed in a container that uses compressed gas, a haloalkane, chloroflourocarbons, volatile hydrocarbons, dimethyl ether, methyl ether, nitrous oxide, carbon dioxide, hydrofluoroalkanes, 1,1,1,2,-tetrafluoroethane (HFA 134a), 1,1,1,2,3,3,3-heptafluoropropane (HFA 227), and combinations thereof, to deliver the composition or mixture. In embodiments, the compressed gases can be considered as the application material. In such embodiments, the application material container can serve as both the application material container and the pressure delivery unit. As will be evident, in embodiments where the compressed gas serves as the application material and it is present with the composition, the composition container or the application material container can serve the function as composition container, application material container, and pressure delivery unit.

In certain embodiments, the composition may be housed in a pre-mix compartment and the application material may be mixed in a separate secondary pre-mix compartment, where the two are separated by a barrier that forms the two compartments. When the user is ready to mix the composition and the application material, the separation barrier may be removed, punctured, sheared, etc. In some embodiments, the separation barrier may be a key, a pin, or a panel or passageway that may be sheared or activated.

In exemplary embodiments, the composition or mixture exits the device, and in particular the application unit, through a nozzle. While not limited in size or shape, in certain embodiments, the nozzle may have holes in the range of 0.05 mm-100 mm, or in the range of 0.05 mm-50 mm, 0.05 mm-25 mm, 0.05 mm-10 mm, and 0.05 mm-1 mm. In certain embodiments, a nozzle with holes having 0.6 mm may be used. In some embodiments, an airbrush-type device having a nozzle with holes of 0.6 mm may be used but, for example and without limitation, an airbrush-type device may have a nozzle having holes in the range of 0.2 mm-10 mm. In certain embodiments, an airbrush equipped with compressed air may be used to facilitate rapid delivery and may allow for higher surface area coverage. In one embodiment, the IWATA Eclipse HP-BCS Bottom Feed Airbrush may be used for spraying powder and liquids. In some embodiments, the nozzle size is about 0.03 mm-1 mm and the nozzle may be configured for inhalation, aerosolization, or nasal applications.

While in some embodiments that relate to mixing of the composition and application material, mixing occurs in a container or the application unit, in other embodiments, the composition may be mixed with the application material at a nozzle to produce an application mixture. In an embodiment, mixing at the nozzle may take place while spraying the composition when the platelet material is in the form of a powder.

In an embodiment, multiple nozzles may be arranged in an array or nozzle bank that may be able to supply a composition or a mixture of the composition and application material, especially those in the form of powders, on all sides of a standing or recumbent patient, which allows for application of the composition or a mixture of the composition and the application material to essentially all available surfaces of a human body. In an embodiment, this may take the form of a bank of nozzles positioned on all sides of a patient to simultaneously cover all surfaces of the patient while standing or laying in an enclosed area, which may take the form of a shower. In another embodiment, there may be a single nozzle or there may be a bank of nozzles facing in the same direction. When positioning a bank of nozzles surrounding a patient (or caregiver who might want to clean the surfaces of his or her protective clothing), the nozzles may be spaced about 50 mm-1000 mm from the patient or caregiver. In one embodiment, where the nozzles are arranged in a shower fashion, the entire enclosure may be in the range of about 1-10 meters wide, about 1-10 meters long, and about 1-10 meters high.

The nozzle hole patterns are not particularly limited. In some embodiments, the nozzles are round, square, rectangular, or atomizer-type. The nozzle spray patterns are also not particularly limited. In an embodiment, the spray patterns are in the forms of a cone, full cone, hollow cone, misting, atomizing, fan, spiral, etc.

The composition, optional application material, and device of the invention can be advantageously used to deliver biologically active substances to subjects. In particular, methods of delivery (also referred to herein as administration) can provide therapeutic and prophylactic treatments for patients and healthcare workers who are exposed to blood or blood-borne pathogens and disease states. In general, the methods can be considered methods of treating a patient or healthcare worker who is bleeding or is expected to come in contact with blood to stop the bleeding or protect from harm from blood.

Stated another way, the composition of the invention can be used in a method of treating a subject. Embodiments of the method comprise administering the composition to a subject in need of platelets or one or more platelet functions. An advantage of the present method is that it can be performed to treat injuries, wounds, or diseases involving bleeding via administration to a patient in need by direct application (such as by topical administration) rather than as an infusion. In embodiments, the method of treating can be thought of as use of the composition of the invention in the treatment of bleeding or a disease that is associated with bleeding.

In an embodiment, the composition may be administered using a nebulizer, aerosolizer, vaporizer, or the like. The composition can be applied to the patient directly, or sprayed onto bandages or equipment that might come in contact with the patient. In some embodiments, the composition can be injected or sprayed inside of a patient's nostrils or other orifices. In an embodiment, the composition may be administered through inhalation. In an embodiment, the composition may be sprayed or otherwise applied onto gauze, a sponge, a bandage, or the like, which may then be applied to a wound. In an embodiment, a wound dressing may include the composition and be bioabsorbable, and thus not require any dressing changes. In an embodiment, spraying or wound dressing materials may include a concentrated platelet material within the composition that may allow for faster healing. In an embodiment, wound dressings may be formulated into about 5 cm by 5 cm pads, or about 10 cm by 10 cm pads, or about 2.5 cm diameter, or about 5 cm diameter pads. The wound can be any wound on a bodily surface, including skin, mucous membranes, oral surfaces, etc.

In an embodiment, the composition is used to treat burns, lacerations, ocular, internal, or oral injuries, and may be utilized during surgery to control bleeding, or assist in post-surgical healing. In certain embodiments, the composition applied directly to lacerations or other openings created during surgical procedures. In one embodiment, the composition is delivered to blood covered surfaces. In another embodiment, the composition can be sprayed to entirely cover the skin of a patient to suppress any future bleeding. In certain embodiments, a coating of the composition can produce hemostatic effects for at least or about two or three days, preferably at least or about five to ten days, or most preferably for up to about 14 days.

In some embodiments, a platelet containing or derived solution or powder can be applied to a patient's skin using air driven or airless sprayers, where the application can encase the individual in a protective layer. This layer not only inhibits bleeding in the patient and initiates a barrier to transmission of any blood-borne diseases, but just as importantly protects healthcare workers or other parties from the blood or its contents, including infectious viruses or bacteria. In this manner the patient can more safely be exposed to non-infected individuals for transportation or routine work in a healthcare center. In certain embodiments, the composition can automatically be applied to a patient at thresholds of rooms, such as surgical rooms.

### EXAMPLES

The invention will be further explained by the following Examples, which are intended to be purely exemplary of the invention, and should not be considered as limiting the invention in any way.

In an experimental application of embodiments disclosed herein, lyophilized platelets were reduced into a fine powder. The powdered platelet material was placed into an IWATA Eclipse HP-BCS Bottom Feed Airbrush with an air compressor for spraying powder and liquids, with a 0.6 mm orifice. The airbrush was used to dispense platelet material in the form of powder onto a pre-wetted surface while varying the air pressure. Varying the pressure allows one to alter the configuration of the spray during application of the platelets.

In another experimental application of embodiments disclosed herein, lyophilized platelets were reyhdrated with water, and the rehydrated platelet material was placed into an airbrush. A highly concentrated mixture of platelet material was sprayed onto both wet and dry surfaces. After about 5-10 minutes, a dry glaze developed. This result shows that a coating of platelet or platelet-derived material can be applied to a surface to create a film, which can be therapeutic or prophylactic.

In another experimental application of embodiments disclosed herein, lyophilized platelets were reduced into a fine powder. The powdered platelet material was dispensed into a small manual atomizer and nebulizer, which was puffed or sprayed onto a wet surface. The powder was absorbed, creating a wet layer containing rehydrated powder. The wet layer dried in about 1-3 minutes.

It will be apparent to those skilled in the art that various modifications and variations can be made in the practice of the present invention and in construction of the device without departing from the scope or spirit of the invention. One having ordinary skill in the art will readily understand that the invention as described above may be practiced with steps in a different order, and/or with hardware elements in configurations that are different than those specifically disclosed. It is intended that the reader understands that features described in connection with one embodiment of the invention may be used in conjunction with other embodiments, even if not explicitly stated above. In addition, other embodiments of the invention will be apparent to those skilled in the art from consideration of the specification and practice of the invention. It is intended that the specification and examples be considered as exemplary only, with a true scope and spirit of the invention being indicated by the following claims.

1. A system for applying platelets or platelet-derived material to a surface, said system comprising a device comprising, in physical and fluid connection, at least one pressure delivery unit, at least one composition container, and at least two application units;
wherein the composition container contains a composition comprising the platelets or platelet-derived material, and
wherein the at least two application units each contain at least one nozzle, forming an array or nozzle bank.

2. The system of claim 1, wherein the pressure delivery unit delivers pressure by way of a pneumatic pump.

3. The system of claim 1, wherein the pressure delivery unit delivers pressure by way of a gas or liquid propellant.

4. The system of claim 1, wherein the pressure delivery unit delivers pressure by way of compressive force supplied by a human.

5. The system of claim 1, wherein the composition comprises lyophilized platelets.

6. The system of claim 5, wherein the lyophilized platelets are in a dry state.

7. The system of claim 5, wherein the lyophilized platelets are in a rehydrated state, and wherein the composition further comprises platelet-derived material.

8. The system of claim 1, wherein the composition comprises one or more therapeutic agents, one or more disinfectants, or both.

9. The system of claim 1, wherein the surface is a surface on a human body.

10. The system of claim 1, wherein the surface is a surface of a piece of medical equipment.

11.-13. (canceled)

14. The system of claim 1, wherein the nozzles are configured to cause application of the composition to essentially all available surfaces of a human body.

15. A method of treating bleeding in a subject or protecting a subject from harmful effects of such bleeding, said method comprising using the system of claim 1 to apply the composition to a surface where bleeding is occurring or where blood from said bleeding is expected to be found.

16. The method of claim 15, wherein the surface is a surface on the body of a human.

17. The method of claim 16, wherein the surface comprises a site where blood is present.

18. The method of claim 15, wherein the surface is a surface of a piece of medical equipment.

19. The method of claim 15, wherein said bleeding includes blood containing an infectious agent.

20. The method of claim 19, wherein the infectious agent is one that causes a hemorrhagic fever.

21. The system of claim 1, further comprising an application material container, wherein the application material container contains application material that improves the delivery, maintenance, or both, of the platelets or platelet-derived material at a desired site on the surface.

22. The system of claim 21, wherein the composition and application material mix at said at least two nozzles.
